(19) United States
(12) Patent Application Publication
(10) Pub. No.: US 2022/0305090 A1
(43) Pub. Date: Sep. 29, 2022

(54) HAIR GROWING AGENT AND FOOD OR BEVERAGE PRODUCT COMPRISING SAME

(71) Applicant: NITTA GELATIN INC., Osaka-shi, Osaka (JP)

(72) Inventors: Seiko KOIZUMI, Yao-shi, Osaka (JP); Hiroshi MANO, Iruma-gun, Saitama (JP); Jun SHIMIZU, Sakado-shi, Saitama (JP); Yoshifumi KIMIRA, Katsushika-ku, Tokyo (JP); Kaho NOMURA, Misato-shi, Saitama (JP)

(73) Assignee: NITTA GELATIN INC., Osaka-shi, Osaka (JP)

(21) Appl. No.: 17/606,115

(22) PCT Filed: Jul. 13, 2020
(86) PCT No.: PCT/JP2020/027260

§ 371 Date: Oct. 25, 2021

(30) Foreign Application Priority Data
Jul. 25, 2019 (JP) 2019-137125

(51) Int. Cl. A61K 38/39; A61P 17/14; A61K 31/401; A61K 38/05; A61K 38/06; A23L 33/175; A23L 33/18

(52) U.S. Cl. CPC A61K 38/39; A61P 17/14; A61K 31/401; A61K 38/05; A61K 38/06; A23L 33/175; A23L 33/18; A23V 2002/00

(57) ABSTRACT

A hair growing agent comprises one or more amino acids or peptides selected from the group consisting of Hyp, Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly, a salt thereof, or a chemically modified product thereof.

### TECHNICAL FIELD

The present invention relates to a hair growing agent, and a food or beverage product containing the same.

### BACKGROUND ART

Collagen hydrolysates (hereinafter, also referred to as “collagen peptide mixtures”) are known to exhibit various physiological activities on living organisms. For example, WO 2012/102308 (PTL 1) discloses that a collagen peptide mixture is used as a therapeutic or prophylactic agent for diabetes because the collagen peptide mixture has an action on enzymes which control insulin secretion. Japanese Patent Laying-Open No. 2009-120512 (PTL 2) discloses that a collagen peptide mixture is used as an articular cartilage regeneration promoter because the collagen peptide mixture has a cartilage regeneration promoting action. Japanese Patent Laying-Open No. 2005-029488 (PTL 3) discloses that a collagen peptide mixture is used as a blood-pressure lowering agent because the collagen peptide mixture has a blood-pressure lowering action.

### CITATION LIST

### Patent Literature

PTL 1: WO 2012/102308

PTL 2: Japanese Patent Laying-Open No. 2009-120512

PTL 3: Japanese Patent Laying-Open No. 2005-029488

PTL 4: Japanese Patent Laying-Open No. 2009-161509

### Non Patent Literature

NPL 1: Tanimura S et al., Cell Stem Cell, 2011, Vol 8, pp. 177-187

### SUMMARY OF INVENTION

### Technical Problem

Here, Japanese Patent Laying-Open No. 2009-161509 (PTL 4) and NPL 1 disclose that XVII-type collagen has a hair loss suppressive action and a hair depigmentation suppressive action, but it has not been heretofore known that the above-described collagen peptide mixture has a promoting action on hair development or hair growth in the hair of head or a hair loss progression preventing action. Thus, studies have been extensively conducted for exploring a promoting action on hair development or hair growth in the hair of head or a hair loss progression preventing action as new physiological activity of collagen peptide mixtures and collagen-derived amino acids, peptides and the like contained in the collage peptide mixtures.

In view of the circumstances described above, an object of the present invention is to provide a hair growing agent comprising an amino acid, a peptide or the like which exhibits at least one of a promoting action on hair development or hair growth in the hair of head or a hair loss progression preventing action, and a food or beverage product comprising the hair growing agent.

### Solution to Problem

In exploration of new physiological activity of a collagen peptide mixture, the present inventors have found that a predetermined amino acid, a predetermined peptide and the like contained in the collagen peptide mixture exhibit at least one of a promoting action on hair development or hair growth in the hair of head or a hair loss progression preventing action, and thus the present invention has been achieved. Specifically, the present invention is as follows.

A hair growing agent according to the present invention comprises one or more amino acids or peptides selected from the group consisting of Hyp, Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly, a salt thereof, or a chemically modified product thereof.

Preferably, the amino acids and the peptides are derived from collagen.

Preferably, the hair growing agent is a collagen peptide mixture comprising at least one of the amino acids or the peptides.

Preferably, the collagen peptide mixture has a weight average molecular weight of 100 Da or more and 8,000 Da or less.

Preferably, the hair growing agent is a cell growth promoter for hair papilla cells.

Preferably, the hair growing agent is a promoter of hair development or hair growth in the hair of head, or a hair loss progression preventing agent.

The food or beverage product according to the present invention comprises the hair growing agent.

### Advantageous Effects of Invention

According to the present invention, it is possible to provide a hair growing agent comprising an amino acid, a peptide or the like which exhibits at least one of a promoting action on hair development or hair growth in the hair of head and a hair loss progression preventing action, and a food or beverage product comprising the hair growing agent.

### BRIEF DESCRIPTION OF DRAWINGS

FIG. 1 is a photographic diagram showing a head of a 11-week-old hairless mouse in a control group given a magnesium-deficient specialty feed.

FIG. 2 is a photographic diagram showing a head of a 11-week-old hairless mouse in a first group given a magnesium-deficient specialty feed containing Pro-Hyp.

### DESCRIPTION OF EMBODIMENTS

Hereinafter, embodiments of the present invention will be described in more detail. As used herein, the notation in the form of “A to B” means the upper limit and the lower limit of a range (i.e. A or more and B or less), and when a unit is not described for A, and a unit is described only for B, the unit for A is identical to the unit for B. As used herein, the term “hair growth” in the “hair growing agent” includes not only the meaning of “hair growth” indicating an action of growing hair, but also the meaning of “hair development” indication an action of developing new hair and promoting the growth of the hair, and the meaning of “prevention of progression of hair loss” indicating an action of reducing the possibility of losing hair.

### [Hair Growing Agent]

The hair growing agent according to the present invention contains one or more amino acids or peptides selected from the group consisting of Hyp, Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly, a salt thereof, or a chemically modified product thereof The hair growing agent having such a characteristic has a hair papilla cell growth promoting action, and therefore can exhibit at least one of a promoting action on hair development or hair growth in the hair of head and a hair loss progression preventing action.

[Predetermined Amino Acid or Predetermined Peptide Exhibiting Promoting action on Hair Development or Hair Growth in Hair of Head or Preventing Progression of Hair Loss, or Salt Thereof, or Chemically Modified Product Thereof]

As described above, the hair growing agent comprises one or more amino acids or peptides selected from the group consisting of Hyp, Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly, a salt thereof, or a chemically modified product thereof In the present description, the “amino acid” is represented by a three-character abbreviation unless otherwise specified. Further, the “amino acid” means an L-type amino acid unless otherwise specified. For the “peptide” in the present description, for example, “Pro-Hyp” means a peptide (dipeptide) in which proline and hydroxyproline are arranged in this order from the N-terminal side toward the C-terminal side, and “Glu-Hyp-Gly” means a peptide (tripeptide) in which glutamic acid, hydroxyproline and glycine are arranged in this order from the N-terminal side toward the C-terminal side. The same applies to the descriptions of dipeptides and tripeptides other than “Pro-Hyp” and “Glu-Hyp-Gly”.

Preferably, the hair growing agent comprises one or more amino acids or peptides selected from the group consisting of Hyp, Pro-Hyp, Hyp-Gly, Gly-Pro, Pro-Ala, Pro-Gly, Pro-Pro, Gly-Pro-Hyp, Ala-Hyp-Gly, Glu-Hyp-Gly and Pro-Ala-Gly, a salt thereof, or a chemically modified product thereof. More preferably, the hair growing agent comprises at least one of the peptides of Pro-Hyp or Hyp-Gly, a salt thereof, or a chemically modified thereof Further, the hair growing agent may comprise a combination of Hyp and Pro, or a combination of Hyp and Gly. In such a case, the hair growing agent can more markedly exhibit a hair papilla cell growth promoting action.

The “salts” of the above-described amino acids and peptides are formed as, for example, inorganic acid salts such as hydrochlorides, sulfates and phosphates, organic acid salts such as methanesulfonates, benzenesulfonates, succinates and oxalates, inorganic base salts such as sodium salts, potassium salts and calcium salts and organic base salts such as triethylammonium salts, of the above-described amino acid or peptides.

The “chemically modified product” of each of the amino acids and peptides means a compound in which a free functional group of an amino acid residue that is a constituent unit is chemically modified. Chemical modification can be performed on, for example, a hydroxyl group of hydroxyproline, an amino group of an amino acid on the N-terminal (amino terminal) side and a carboxyl group of an amino acid on the C-terminal (carboxyl terminal) side. For specific means and treatment conditions for chemical modification, known conventional chemical modification techniques targeting amino acids and peptides are applied. The chemically modified product of each of the amino acids and peptides, which is obtained by such chemical modification, can produce an enhancing effect on solubility under a mildly acidic to neutral condition, an enhancing effect on compatibility with other active ingredients, and the like.

For example, the tripeptide of Glu-Hyp-Gly can be subjected to O-acetylation as chemical modification of a hydroxyl group in hydroxyproline. The O-acetylation can be performed by applying acetic anhydride to the peptide in an aqueous solvent or a nonaqueous solvent. Esterification, amidation or the like can be performed as chemical modification of a carboxyl group in glycine. The esterification can be performed by suspending the peptide in methanol, and then causing dry hydrogen chloride gas to pass through the resulting suspension. The amidation can be performed by applying carbodiimide or the like to the peptide.

Methylation can be performed as chemical modification of a free amino group in the peptide. At least one of phosphorylation and sulfation can be performed as chemical modification of a free hydroxyl group in the peptide.

Preferably, the amino acid and peptide are derived from collagen. Here, the collagen as a raw material can be obtained by performing known conventional defatting or decalcification treatment, extraction treatment or the like on, for example, the skin, the dermis, the bone, the cartilage, the tendon or the like of animals typically of a bovine, a pig, a sheep, a chicken or an ostrich, or the bone, the skin, the scale or the like of fish. Further, gelatin can be used as a raw material for the peptide. The gelatin can be obtained by treating the thus-obtained collagen through a known conventional method such as extraction with hot water. For the collagen and the gelatin, commercial products can be used as raw materials.

The amino acid and peptide can be obtained by hydrolyzing the collagen and/or the gelatin with two or more of endo-type proteases and exo-type proteases in combination. The amino acid and peptide can be obtained as a collagen peptide mixture which exists together with other collagen peptides due to the hydrolysis, but any of the collagen peptide mixture itself and a mixture obtained by partially purifying the collagen peptide mixture can be used as the hair growing agent according to the present invention. That is, it is also preferable that the hair growing agent be a collagen peptide mixture comprising at least one of the above-described amino acids or peptides. Further, by further purifying the collagen peptide mixture, a purified product comprising one of the above-described amino acids and peptides can be obtained with a high purity. When the amino acid and peptide are derived from collagen, it is preferable to obtain the amino acid and peptide by using a method in which collagen or gelatin is enzyme-treated in two stages as described below.

Further, the collagen peptide mixture preferably has a weight average molecular weight of 100 Da or more and 8,000 Da or less. The weight average molecular weight of the collagen peptide mixture is more preferably 100 Da or more and 6,000 Da or less, still more preferably 100 Da or more and 4,000 Da or less. When the weight average molecular weight of the collagen peptide mixture is within the above-described range, the hair growing agent more markedly exhibits a hair papilla cell growth promoting action, and therefore it is possible to sufficiently obtain at least one of a promoting action on hair development and hair growth in the hair of head and a hair loss progression preventing action. If the weight average molecular weight is more than 8,000 Da, the above-described effects of the hair growing agent may be insufficient.

The weight average molecular weight of the collagen peptide mixture can be determined by carrying out size exclusion chromatography (SEC) under the following measurement conditions.

Equipment: High-performance liquid chromatography (HPLC) (manufactured by TOSOH CORPORATION)

Column: TSKGe1 (registered trademark) G2000SW_{XL}

Column temperature: 40° C.

Column size: 7.8 mm (I.D.)×30 cm, 5 μm

Eluant: 45 mass % acetonitrile (with 0.1 mass % trifluoroacetic acid)

Flow rate: 1.0 mL/min

Injection amount: 10 μL

Detection: UV 214 nm

Molecular weight marker: The following five types are used
Cytochrome C Mw: 12,000
Aprotinin Mw: 6,500
Bacitracin Mw: 1,450
Gly-Gly-Tyr-Arg Mw: 451
Gly-Gly-Gly Mw: 189

Specifically, a sample containing about 0.2 g of the collagen peptide mixture is added to about 100 ml of distilled water, the mixture is stirred, and then filtered with a 0.2 μm filter to prepare a sample of which weight average molecular weight is measured (measurement specimen). By subjecting the measurement specimen to the size exclusion chromatography, the weight average molecular weight of the collagen peptide mixture can be determined.

### [Method for Producing Hair Growing Agent]

The amino acid or peptide contained in the hair growing agent can be obtained by known conventional methods. For example, the amino acid (Hyp) can be obtained by purchasing a commercially available amino acid. The amino acid can also be obtained by using a method including hydrolyzing collagen or gelatin.

The peptides (Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly) can be each obtained by using a known conventional liquid-phase or solid-phase peptide synthesis method, or a method including hydrolyzing collagen or gelatin. From the viewpoint of efficiency, it is preferable to produce the peptide by using a chemical synthesis method using an amino acid as described below, or a method including enzymatically treating collagen or gelatin in two stages as described below. Further, the peptide can be produced by using a method including performing enzymatic treatment with only a secondary enzyme with a primary enzyme omitted, or a method including performing enzymatic treatment with a primary enzyme and a secondary enzyme simultaneously, instead of the method including enzymatically treating collagen or gelatin in two stages. Hereinafter, a method for producing, in particular, “Glu-Hyp-Gly”, among the peptides contained in the hair growing agent, will be described as an example of a method for producing a peptide contained in the hair growing agent.

### <Chemical Synthesis Method>

The peptide can be obtained by using a common peptide synthesis method. As the peptide synthesis method, a solid-phase synthesis method and a liquid-phase synthesis method are known. As the solid-phase synthesis method, an Fmoc method and a Boc method are known. The peptide can be obtained by using either of the Fmoc method and the Boc method. As the solid-phase peptide synthesis method, a method for synthesizing a tripeptide represented by Glu-Hyp-Gly can be carried out as follows.

First, a bead of a polystyrene polymer gel having a diameter of about 0.1 mm and having a surface modified with amino groups is provided as a solid phase. Separately, diisopropylcarbodiimide is provided as a condensing agent. Next, the amino group of glycine, which is an amino group on the C-terminal (carboxyl terminal) side in the amino acid sequence, is protected with an Fmoc (fluorenyl-methoxy-carbonyl) group, the carboxyl group of the glycine is peptide-bound to the amino group as the solid phase through a dehydration reaction using the condensing agent. Further, the solid phase is washed with a solvent to remove the remaining condensing agent and amino acids, followed by removing the protecting group (deprotecting) of the amino group of glycine which is peptide-bound to the solid phase.

Subsequently, hydroxyproline in which an amino group is protected with an Fmoc group is provided, and the carboxyl group of the hydroxyproline is peptide-bound to the deprotected amino group of the glycine by using the condensing agent. Thereafter, in the same manner as described above, the amino group of the hydroxyproline is deprotected, glutamic acid protected with an Fmoc group is provided, and a reaction for peptide-binding the glutamic acid to the hydroxyproline is carried out to synthesize a tripeptide represented by Glu-Hyp-Gly as the solid phase. Finally, the tripeptide can be produced by deprotecting the amino group of the glutamic acid, and separating the tripeptide from the solid phase by immersion in trifluoroacetic acid under heating.

### <Production Method Using Collagen and Gelatin>

Further, a method for enzymatically treating collagen or gelatin in two stages to produce a tripeptide represented by Glu-Hyp-Gly can be carried out as follows.

The term “enzymatically treating (collagen or gelatin) in two stages” means the following. That is, primary enzymatic treatment is performed by a known conventional method for breaking the peptide bond of collagen or gelatin, and secondary enzymatic treatment is then performed with an enzyme having aminopeptidase N activity, an enzyme having both aminopeptidase N activity and prolyl tripeptidyl aminopeptidase activity, or a combination of an enzyme having aminopeptidase N activity and an enzyme having prolyl tripeptidyl aminopeptidase activity. By performing the primary enzymatic treatment, a collagen peptide mixture precursor can be obtained. By further performing the secondary enzymatic treatment, a collagen peptide mixture containing the Glu-Hyp-Gly can be obtained from the collagen peptide mixture precursor. The method for enzymatically treating collagen or gelatin in two stages will be described in more detail below.

### (Primary Enzymatic Treatment)

The enzyme used in the primary enzymatic treatment should not be particularly limited as long as it is an enzyme capable of breaking peptide bonds of collagen or gelatin, and any proteolytic enzyme can be used. Specifically, examples of thereof include collagenase, thiol protease, serine protease, acidic protease, alkaline protease and metal protease. One selected from the group consisting of these enzymes may be used alone, or two or more thereof may be used in combination. As the thiol protease, chymopapain, papain, bromelain and ficin derived from plants, cathepsin and calcium dependent protease derived from animals, and the like can be used. As the serine protease, trypsin, cathepsin D and the like can be used. As the acidic protease, pepsin, chymotrypsin and the like can be used. Considering that the hair growing agent according to the present invention is used for medicaments, specified health food and the like, it is preferable that as the enzymes used in the primary enzymatic treatment, those other than enzymes derived from pathogenic microorganisms be used.

The amount of enzymes in the primary enzymatic treatment is, for example, preferably 0.1 to 5 parts by mass of the above-described enzymes based on 100 parts by mass of collagen or gelatin. Preferably, the treatment temperature and the treatment time in the primary enzymatic treatment are 30 to 65° C. and 10 minutes to 72 hours, respectively. The weight average molecular weight of the collagen peptide mixture precursor obtained through the primary enzymatic treatment is preferably 500 to 20,000 Da, more preferably 500 to 10,000 Da, still more preferably 500 to 8,000 Da. It can be said that when the weight average molecular weight is within the above-described range, a peptide having an appropriate molecular weight is adequately generated. If necessary, the enzyme can be deactivated after the primary enzymatic treatment. In this case, the deactivation temperature is, for example, preferably 70 to 100° C. The weight average molecular weight of the collagen peptide mixture precursor can be determined by the method using SEC.

### (Secondary Enzymatic Treatment)

Examples of the enzyme used in the secondary enzymatic treatment include enzymes having aminopeptidase N activity, enzymes having both aminopeptidase N activity and prolyl tripeptidyl aminopeptidase activity, and combinations of an enzyme having aminopeptidase N activity and prolyl tripeptidyl aminopeptidase activity. The term “enzyme having aminopeptidase N activity” as used herein is a peptidase having a function of releasing an amino acid from the N-terminal side of the peptide chain, where the enzyme acts when an amino acid other than proline or hydroxyproline exists at the second position from the N-terminal side. The term “enzyme having prolyl tripeptidyl aminopeptidase activity” as used herein is a peptidase which releases only three amino acid residues on the N-terminal side from a peptide having proline or hydroxyproline at the third position from the N-terminal side. Considering that the hair growing agent according to the present invention is used for medicaments, specified health food and the like, it is preferable that as the enzymes used in the secondary enzymatic treatment, those other than enzymes derived from pathogenic microorganisms be used.

Examples of the enzyme having aminopeptidase N activity include aminopeptidase N (EC 3.4.11.2.; T. Yoshimoto et al., Agric. Biol. Chem., 52: 217-225 (1988)), and enzymes having aminopeptidase N activity derived from Aspergillus. Examples of the enzyme having prolyl tripeptidyl aminopeptidase activity include prolyl tripeptidyl aminopeptidase (EC 3.4.14.; A. Banbula et al., J. Biol. Chem., 274: 9246-9252 (1999)).

By performing the secondary enzymatic treatment, a collagen peptide mixture containing a peptide which has not been contained in the collagen peptide mixture precursor can be obtained. Specifically, a collagen peptide mixture containing the Glu-Hyp-Gly can be obtained.

The amount of enzymes in the secondary enzymatic treatment is, for example, preferably 0.01 to 5 parts by mass of the above-described enzymes based on 100 parts by mass of the collagen peptide mixture precursor. Preferably, the treatment temperature and the treatment time in the secondary enzymatic treatment are 30 to 65° C. and 10 minutes to 72 hours, respectively. The weight average molecular weight of the collagen peptide mixture obtained through the secondary enzymatic treatment is preferably 100 to 10,000 Da, more preferably 100 to 8,000 Da, still more preferably 100 to 4,000 Da. The weight average molecular weight of the collagen peptide mixture can be determined by the method using SEC.

The secondary enzymatic treatment is performed mainly for the purpose of generating the tripeptide of Glu-Hyp-Gly. Thus, it is preferable to adjust the amount of enzymes, the treatment temperature, the treatment time and the pH in the secondary enzymatic treatment so that the peptide contained in the collagen peptide mixture precursor is not excessively hydrolyzed. Accordingly, the weight average molecular weight of the collagen peptide mixture is preferably within the above-described range. It is necessary to deactivate the enzyme after the secondary enzymatic treatment. In this case, the deactivation temperature is, for example, preferably 70 to 100° C. Further, it is preferable to perform sterilization treatment at 120° C. for several seconds or more. In addition, the collagen peptide mixture can be subjected to spray drying by applying heat at 200° C. or higher.

In the secondary enzymatic treatment, not only the enzymes having aminopeptidase N activity and enzymes having prolyl tripeptidyl aminopeptidase activity, but also enzymes having different activities can be used, and two or more enzymes each having different activities can be used in combination. Consequently, by-products can be digested and removed. Preferably, the enzymes used in this case are appropriately selected, depending on the type of collagen used as a raw material, and the type of enzyme used in the primary enzymatic treatment. Examples of the different activities include dipeptidase activity such as prolidase activity and hydroxyprolidase activity. Consequently, by-products such as dipeptides can be digested and removed.

Further, the aminopeptidase N activity is basically activity causing the release of amino acids on the N-terminal side one by one. Thus, when the secondary enzymatic treatment is performed only with an enzyme having aminopeptidase N activity in the case where the collagen peptide mixture precursor obtained through the primary enzymatic treatment contains a peptide having an extremely large molecular weight, the duration for the secondary enzymatic treatment markedly increases. For coping with such a case, for example, prolyl oligopeptidase which is an endopeptidase having activity causing hydrolysis of proline on the carboxyl group side (prolidase activity) can be used in the secondary enzymatic treatment. Consequently, the secondary enzymatic treatment can be efficiently performed.

In the method including enzyme-treating collagen or gelatin in two stages, the primary enzymatic treatment enables generation of a peptide having a relatively large molecular weight. This peptide can have an amino acid sequence represented by, for example, [X₁-Gly-X₂-Glu-Hyp-Gly] (X₁ and X₂≠Hyp). In the subsequent secondary enzymatic treatment, an enzyme having aminopeptidase N activity acts on the peptide represented by [X₁-Gly-X₂-Glu-Hyp-Gly], so that X₁ at the N-terminal is released to obtain a peptide having an amino acid sequence represented by [Gly-X₂-Glu-Hyp-Gly]. Next, an enzyme having aminopeptidase N activity acts twice on the peptide represented by [Gly-X₂-Glu-Hyp-Gly], so that glycine and X₂ are released to obtain a peptide represented by [Glu-Hyp-Gly].

### (Purification of Collagen Peptide Mixture)

By performing enzymatic treatment in two stages as described above, a collagen peptide mixture containing Glu-Hyp-Gly can be produced. Since the collagen peptide mixture contains peptides other than the tripeptide represented by Glu-Hyp-Gly, it is preferable to purify the collagen peptide mixture if necessary. As a purification method in this case, a known conventional method can be used, and examples thereof include ultrafiltration, and various types of liquid chromatography such as size exclusion chromatography, ion-exchange chromatography, reversed phase chromatography and affinity chromatography.

Specifically, the collagen peptide mixture can be purified in accordance with the following procedure. That is, about 2 g/10 ml of the collagen peptide mixture is loaded into an ion-exchange column (e.g. “TOYOPEARL” (registered trademark) DEAE-650″ (trade name) manufactured by TOSOH CORPORATION), and a first void volume fraction eluted with distilled water is then collected. Subsequently, the first void volume fraction is loaded into a column having an ion-exchange group opposite to that of the above ion-exchange column (e.g. “TOYOPEARL” (registered trademark) SP-650 manufactured by TOSOH CORPORATION), and a second void volume fraction eluted with distilled water is then collected.

Next, the second void volume fraction is loaded into a gel filtration column (e.g. “SEPHADEX LH-20” (trade name) manufactured by GE Healthcare Japan

Corporation), and eluted with a 30 mass % methanol aqueous solution to collect a fraction containing the tripeptide of Glu-Hyp-Gly. Finally, using a high-performance liquid chromatography (HPLC) with a reversed-phase column (e.g. “Pondasphere 5μ C18 300 Å Column” (trade name) manufactured by Waters Corporation), the fraction is fractionated in accordance with a linear concentration gradient of a 32 mass % or less acetonitrile aqueous solution containing 0.1 mass % trifluoroacetic acid. In this way, Glu-Hyp-Gly can be obtained with a high purity.

### [Cell Growth Promoter for Hair Papilla Cells, Promoter of Hair Development or Hair Growth in Hair of Head and Hair Loss Progression Preventing Agent]

The hair growing agent according to the present invention is preferably a cell growth promoter for hair papilla cells. As described above, the hair growing agent comprises one or more amino acids or peptides selected from the group consisting of Hyp, Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly, a salt thereof, or a chemically modified product thereof, and therefore can exhibit a hair papilla cell growth promoting action. This enables the hair growing agent to exhibit at least one of a promoting action on hair development or hair growth in the hair of head and a hair loss progression preventing action. Thus, the hair growing agent can be used for the purpose of promoting cell growth of hair papilla cells as a cell growth promoter for hair papilla cells.

It is also preferable that the hair growing agent be a promoter of hair development or hair growth in the hair of head or a hair loss progression preventing agent because the hair growing agent comprises any of the above-described amino acid or peptides, a salt thereof, or a chemically modified product thereof. As described above, the hair growing agent has a hair papilla cell growth promoting action, and therefore can be used in treatment for promoting hair development or hair growth in the hair of head by growing hair papilla cells as a promoter of hair development or hair growth in the hair of head. Further, the hair growing agent can be used for the purpose of growing hair papilla cells as a hair loss progression preventing agent in the hair of head to suppress and prevent progression of hair loss occurring due to a decrease in hair papilla cells.

The hair growing agent can be orally or parenterally administered in various forms. For these forms, the hair growing agent can take dosage forms such as tablets, granules, capsules, powders, liquids, suspension preparations and emulsion preparations when orally administered. Further, the hair growing agent in any of the above-described dosage forms can be mixed with a food or beverage product. The hair growing agent comprises, for example, at least one of the above-described amino acids, combinations of amino acids or peptides, which are rapidly absorbed in the intestinal tract, and therefore can be orally administered.

When parenterally administered, the hair growing agent can take dosage forms such as external preparations such as ointments, creams and lotions, and transdermal preparations. Further, the hair growing agent can take forms of solutions or coatings to be rubbed into the head skin.

The dose of the hair growing agent varies depending on the age, the sex, the body weight and the sensitivity difference of a subject, the administration method, the administration interval, the type of preparation and the like. When the hair growing agent is orally administered, the dose per adult is, for example, preferably 0.0001 to 2,500 mg/kg, more preferably 0.0001 to 500 mg/kg. When the dosage form of the hair growing agent is, for example, a tablet, the tablet may contain the hair growing agent in an amount of 0.001 to 80 mass % per tablet, and when the dosage form of the hair growing agent is, for example, a powder, the powder may contain the hair growing agent in an amount of 0.001 to 100 mass %. When the hair growing agent is parenterally administered or administered by a preparation in another form, the dose can be appropriately determined by reference to a dose in oral administration. The hair growing agent can be administered daily once or in several divided doses, or administered once every day or every several days.

The hair growing agent may appropriately contain other active ingredients, a preparation carriers and the like as long as the effects of the present invention are not adversely affected. Examples of other active ingredients include inulin, caffeic acid, quinic acid, derivatives thereof, extracts from marjoram, crude drugs such as Kinfukan, milkwort (polygalae radix), Hakubiso and Desmos chinensis Lour, royal jerry, extracts from echinacea, extracts from acai, and extracts from Cupuacu. Further, examples of pharmaceutically acceptable carriers used in formulation into pharmaceutical preparations include diluents, binding agents (syrup, gum arabic, gelatin, sorbitol, tragacanth and polyvinylpyrrolidone), excipients (lactose, sucrose, cornstarch, potassium phosphate, sorbitol and glycine), lubricants (magnesium stearate, talc, polyethylene glycol and silica), disintegrants (potato starch) and wetting agents (sodium lauryl sulfate).

### [Use Invention]

As described above, the hair growing agent according to the present invention comprises one or more amino acids or peptides selected from the group consisting of Hyp, Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly, a salt thereof, or a chemically modified product thereof The hair growing agent has a hair papilla cell growth promoting action as an unknown attribute of the above-described amino acids and peptides, and therefore can exhibit at least one of a promoting action on hair development or hair growth in the hair of head and a hair loss progression preventing action. In other words, the present invention is any of the amino acids or peptides, a salt thereof, or a chemically modified product thereof for promoting hair development or hair growth in the hair of head or preventing hair loss progression.

### [Food or Beverage Product]

The food or beverage product according to the present invention contains the hair growing agent. For example, the peptide, which is preferably contained in the hair growing agent, is rapidly absorbed in the intestinal tract, and therefore can be orally administered. Thus, the hair growing agent of the present invention can be administered as a food or beverage product in which the hair growing agent is mixed with food or a beverage. Further, the food or beverage product according to the present invention can be used as specified health food or food with functional claims. The concentration of the hair growing agent contained in the food or beverage product is preferably 0.001 to 100 mass %.

### EXAMPLES

Hereinafter, the present invention will be described in more detail by way of Example, which should not be construed as limiting the present invention.

### Example 1

### Cell Biological Test (In Vitro Test)

### [Preparation of Sample]

### <Preparation of Amino Acid, Peptide and Collagen Peptide Mixture>

As samples to be used for evaluating a hair papilla cell growth promoting action, amino acids, combinations of amino acids, dipeptides, tripeptides and collagen peptide mixtures shown in Tables 1 and 2 below were provided by production using the above-described methods or purchase from the manufacturers described later. Here, for the amino acids, combinations of amino acids and peptides shown in

Table 1, abbreviations in which amino acids are each represented by one character are used. In Table 1, “PO” represents a dipeptide of proline-hydroxyproline (trade name: “G-3025”, manufactured by BACHEM Co.), and “OG” represents a dipeptide of hydroxyproline-glycine (trade name: “G-2365”, manufactured by BACHEM Co.). “GPO” represents a tripeptide of glycine-proline-hydroxyproline (manufactured by PH Japan Co., Ltd.). “PO” means a peptide in which proline and hydroxyproline are arranged in this order from the N-terminal side toward the C-terminal side. The same applies to the descriptions of peptides other than “PO”.

Further, in Table 1, “AOG” represents a tripeptide of alanine-hydroxyproline-glycine (manufactured by PH Japan Co., Ltd.), and “EOG” represents a tripeptide of glutamic acid-hydroxyproline-glycine (manufactured by PH Japan Co., Ltd.). “SOG” represents a tripeptide of serine-hydroxyproline-glycine (manufactured by PH Japan Co., Ltd.), and “GP” represents a dipeptide of glycine-proline (trade name: “G-3015”, manufactured by BACHEM Co.). “LO” represents a dipeptide of leucine-hydroxyproline (manufactured by PH Japan Co., Ltd.), “FO” represents a dipeptide of phenylalanine-hydroxyproline (manufactured by PH Japan Co., Ltd.), and “EO” represents a dipeptide of glutamic acid-hydroxyproline (manufactured by PH Japan Co., Ltd.).

“PA” represents a dipeptide of proline-alanine (manufactured by PH Japan Co., Ltd.), and “PAG” represents a tripeptide of proline-alanine-glycine (manufactured by PH Japan Co., Ltd.). “PG” represents a dipeptide of proline-glycine (manufactured by PH Japan Co., Ltd.). “PP” represents a dipeptide of proline-proline (manufactured by PH Japan Co., Ltd.). “0” represents hydroxyproline (trade name: “080-01642”, manufactured by FUJIFILM Wako Pure Chemical Corporation), “G” represents glycine (trade name: “073-00732”, manufactured by FUJIFILM Wako Pure Chemical Corporation), “P” represents proline (trade name: “161-04602”, manufactured by FUJIFILM Wako Pure Chemical Corporation), “P+O” represents a combination of the proline and the hydroxyproline, and “O+G” represents a combination of the hydroxyproline and the glycine.

Further, the collagen peptide mixture A (trade name: “TYPE-S”, manufactured by Nitta Gelatin Inc., weight average molecular weight (Mw): about 750 Da) shown in Table 2 was found to include the following composition in quantitative analysis performed by LC-MS/MS under the conditions described later.

Pro-Hyp: 8 ppm, Hyp-Gly: 7,389 ppm, Gly-Pro-Hyp: 8 ppm, Ala-Hyp-Gly: 199 ppm, Glu-Hyp-Gly: 9 ppm, Ser-Hyp-Gly: 176 ppm, Gly-Pro: 1,159 ppm, Pro-Ala-Gly: 2,229 ppm, total: 11,177 ppm.

The collagen peptide mixture B (trade name: “COLLAPEP PU”, manufactured by Nitta Gelatin Inc., weight average molecular weight (Mw): about 630 Da) shown in Table 2 was found to include the following composition in quantitative analysis performed by LC-MS/MS under the conditions described later.

Pro-Hyp: 8 ppm, Hyp-Gly: 3,447 ppm, Gly-Pro-Hyp: 36 ppm, Ala-Hyp-Gly: 436 ppm, Glu-Hyp-Gly: 4 ppm, Ser-Hyp-Gly: 120 ppm, Gly-Pro: 2,379 ppm, Pro-Ala-Gly: 2,645 ppm, total: 9,074 ppm.

The quantitative analysis by LC-MS/MS was performed under the following conditions.

HPLC apparatus: “ACQUITY UPLC H-Class Bio”, manufactured by Waters Corporation)

Column: “Hypersil GOLD PFP 2.1×150 mm, 5 μm (manufactured by Thermo Fisher Scientific. Inc.)

Column temperature: 40° C. (linear gradient)

Mobile phase: (A) aqueous solution containing 0.2% formic acid and 2 mM ammonium acetate
(B) 100% methanol

Gradient Setting

| Time (min) | Flow rate | Mobile phase (mass %) |
|---|---|---|
| Initial | 200 | 98 |
| 3.50 | 200 | 98 |
| 3.51 | 400 | 5 |
| 7.00 | 400 | 5 |
| 7.10 | 200 | 98 |
| 17.00 | 200 | 98 |

Injection amount: 0.5 μl

MS/MS Apparatus: “Xevo TQ-XS” manufactured by Waters Corporation Ionization method: Positive ESI
Capilary (kV): 1
Desolvation temperature (° C.): 500
Source temperature (° C.): 150
MRM conditions:

| Peptide (abbreviation) | precursor ion (m/z) | product ion (m/z) |
|---|---|---|
| Glu-Pro (GP) | 173 | 116 |
| Hyp-Gly (OG) | 189 | 86 |
| Pro-Hyp (PO) | 229 | 132 |
| Ala-Hyp-Gly (AOG) | 260 | 189 |
| Glu-Hyp-Gly (EOG) | 318 | 225 |
| Glu-Pro-Hyp (GPO) | 286 | 155 |
| Ser-Hyp-Gly (SOG) | 276 | 189 |
| Pro-Ala-Gly (PAG) | 244 | 141 |

### <Preparation of Hair Papilla Cells>

First, human normal hair papilla cells HFDPC-C (manufactured by Takara Bio Inc.) were obtained, and the hair papilla cells were then seeded at 0.2×10⁴/dish in each well of a 96-well plate for cell culture (manufactured by Corning Inc.). Further, 200 μL of a basal medium (trade name: “Follicle Dermal Papilla Cell Basal Medium”, manufactured by Takara Bio Inc.) containing a growth factor accompanying the obtained hair papilla cells was supplied to each well, and the hair papilla cells were precultured in each well at 37° C. for 24 hours.

Next, the hair papilla cells were confirmed to be subconfluent, and the basal medium in each well was then replaced by 200 μL of another basal medium (trade name: “Follicle Dermal Papilla Cell Basal Medium”, manufactured by Takara Bio Inc.) free of the growth factor described above. In this way, hair papilla cells to be used for determining whether or not addition of the above-described samples promote cell growth were prepared.

### [Cell Growth Test]

To the hair papilla cells prepared as described above, amino acids, combinations of amino acids, peptides and collagen peptide mixtures which are the samples described above were added at final concentrations shown in Tables 1 and 2, and the hair papilla cells were cultured in each well at 37° C. for 72 hours. Here, to one of the wells containing the hair papilla cells prepared as described above, 20 μL, of purified water was added, and as with other hair papilla cells, culturing was performed at 37° C. for 72 hours to prepare a control test sample (control). Thereafter, for each of the hair papilla cells in the wells containing purified water or the samples, the number of living cells (living cell number) was counted by a neutral red method. Here, the “neutral red method” is a method in which neutral red is added at a final concentration of 150 μg/mL into a well where cells are cultured, the cells are cultured for 20 minutes, and washed with PBS (phosphate buffer physiological saline), 200 μL of a 50 mass % ethanol solution containing 1 mass % acetic acid is added into the well as an extraction liquid, the mixture is stirred, and the absorbance of the well containing the neutral red is measured at a wavelength of 540 nm to measure the number of living cells in the well.

The number of living cells of hair papilla cells in the well containing the sample with respect to the number of living cells of hair pallia cells in the well containing purified water (control test sample) was determined as a cell growth rate (%) to evaluate the hair papilla cell growth promoting action in the sample. Further, the cell growth rate (%) was subjected to statistical processing to evaluate significance of the hair papilla cell growth promoting action in the sample. For the evaluation of significance, statistical processing was performed using software (“Excel (Ver 2016)” (trade name), manufactured by Social Survey Research Information Co., Ltd.), Smirnov-Grubbs (two-sided test) was conducted, and the significance level (P value) was set to 0.05 as a threshold. Thereafter, the Student's t-test (t-test) was conducted to evaluate significance. Tables 1 and 2 show the results. In Tables 1 and 2, samples with “++” were determined to have a significance in the hair papilla cell growth promoting action. In samples with “+”, the cell growth rate (%) exceeded 100.

TABLE 1

| | | | | Ratio to | |
|---|---|---|---|---|---|
| Amino acid | Content (final | | Growth rate | control t- | |
| or peptide | concentration) | | control = 100 | test | Assessment |
| PO | 0.05 | mM | 126 ± 8 | 0.011 | ++ |
| | 0.5 | mM | 127 ± 3 | 0.002 | ++ |
| | 5 | mM | 129 ± 6 | 0.004 | ++ |
| OG | 0.05 | mM | 124 ± 4 | 0.005 | ++ |
| | 0.5 | mM | 121 ± 1 | 0.004 | ++ |
| | 5 | mM | 117 ± 5 | 0.022 | ++ |
| GPO | 0.05 | mM | 126 ± 3 | 0.003 | ++ |
| | 0.5 | mM | 125 ± 4 | 0.003 | ++ |
| | 5 | mM | 120 ± 4 | 0.009 | ++ |
| AOG | 0.05 | mM | 127 ± 5 | 0.003 | ++ |
| | 0.5 | mM | 126 ± 2 | 0.002 | ++ |
| | 5 | mM | 119 ± 2 | 0.006 | ++ |
| EOG | 0.05 | mM | 126 ± 10 | 0.018 | ++ |
| | 0.5 | mM | 131 ± 1 | 0.001 | ++ |
| | 5 | mM | 121 ± 8 | 0.024 | ++ |
| SOG | 0.05 | mM | 113 ± 10 | 0.120 | + |
| | 0.5 | mM | 116 ± 3 | 0.015 | ++ |
| | 5 | mM | 109 ± 2 | 0.084 | + |
| GP | 0.05 | mM | 105 ± 4 | 0.174 | + |
| | 0.5 | mM | 109 ± 2 | 0.005 | ++ |
| | 5 | mM | 114 ± 2 | 0.001 | ++ |
| LO | 0.05 | mM | 104 ± 6 | 0.285 | + |
| | 0.5 | mM | 105 ± 2 | 0.049 | + |
| | 5 | mM | 104 ± 5 | 0.279 | + |
| FO | 0.05 | mM | 104 ± 4 | 0.186 | + |
| | 0.5 | mM | 106 ± 2 | 0.028 | + |
| | 5 | mM | 104 ± 1 | 0.036 | ++ |
| EO | 0.05 | mM | 102 ± 6 | 0.720 | + |
| | 0.5 | mM | 109 ± 4 | 0.070 | + |
| PA | 0.05 | mM | 105 ± 2 | 0.079 | + |
| | 0.5 | mM | 111 ± 1 | 0.002 | + |
| | 5 | mM | 107 ± 5 | 0.094 | + |
| PAG | 0.05 | mM | 110 ± 4 | 0.040 | + |
| | 0.5 | mM | 113 ± 1 | 0.010 | ++ |
| | 5 | mM | 115 ± 4 | 0.010 | ++ |
| PG | 0.05 | mM | 120 ± 5 | 0.005 | ++ |
| | 0.5 | mM | 120 ± 1 | 0.001 | ++ |
| | 5 | mM | 120 ± 5 | 0.004 | ++ |
| PP | 0.05 | mM | 116 ± 3 | 0.004 | ++ |
| | 0.5 | mM | 113 ± 6 | 0.024 | ++ |
| | 5 | mM | 109 ± 2 | 0.019 | ++ |
| O | 0.05 | mM | 114 ± 1 | 0.002 | ++ |
| | 0.5 | mM | 114 ± 3 | 0.006 | ++ |
| | 5 | mM | 110 ± 5 | 0.045 | ++ |
| P + O | 0.05 | mM | 116 ± 3 | 0.003 | ++ |
| | 0.5 | mM | 120 ± 5 | 0.005 | ++ |
| | 5 | mM | 113 ± 2 | 0.005 | ++ |
| O + G | 0.05 | mM | 110 ± 4 | 0.029 | ++ |
| | 0.5 | mM | 110 ± 3 | 0.020 | ++ |
| | 5 | mM | 107 ± 2 | 0.038 | ++ |

TABLE 2

| | Content | | Ratio to | |
|---|---|---|---|---|
| Amino acid | (mass %) (final | Growth rate | control t- | |
| or peptide | concentration) | control = 100 | test | Assessment |
| Collagen | 0.025% | 109 ± 5 | 0.080 | + |
| peptide | 0.050% | 119 ± 4 | 0.003 | ++ |
| mixture A | 0.100% | 127 ± 3 | 0.0003 | ++ |
| Collagen | 0.025% | 109 ± 3 | 0.007 | ++ |
| peptide | 0.050% | 110 ± 2 | 0.001 | ++ |
| mixture B | 0.100% | 111 ± 3 | 0.004 | ++ |

### [Discussions]

From Tables 1 and 2, it is apparent that the one or more amino acids or peptides selected from the group consisting of Hyp, Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly had a hair papilla cell growth promoting action. The collagen peptide mixtures containing the amino acids or peptides had a hair papilla cell growth promoting action. Further, the combination of the amino acids of Pro and Hyp and the combination of the amino acids of Hyp and Gly had a hair papilla cell growth promoting action. This indicates that the above-described amino acids and peptides and collagen peptide mixtures containing the amino acids and peptides were effective as hair growing agents, specifically cell growth promoters for hair papilla cells, promoters of hair development or hair growth in the hair of head, or hair loss progression preventing agents.

### Example 2

### Test for Confirming Hair Development Effect and Hair Growth effect Using Hairless Mouse (In Vivo Test)

Thirty 8-week-old male and female hairless mice were provided by purchase from Hoshino Laboratory Animals, Inc. The hairless mice were divided into five groups each consisting of six mice with no regard to sex. Specifically, the plurality of groups consist of a normal group given a normal feed (trade name: “Labo MR Stock”, manufactured by Nosan Corporation), a control group given magnesium-deficient specialty feed (trade name: “HR-AD Feed”, manufactured by Nosan Corporation), a first group given a mixed feed obtained by adding Pro-Hyp at a content of 0.3 mass % to the magnesium-deficient specialty feed, a second group given a mixed feed obtained by adding the collagen peptide mixture A at a content of 5 mass % to the magnesium-deficient specialty feed, and a third group given a mixed feed obtained by adding a collagen peptide mixture C of the later-described composition at a content of 2.5 mass % to the magnesium-deficient specialty feed.

The collagen peptide mixture C, which is a collagen peptide mixture that is being developed by Nitta Gelatin Inc., was found to include the following composition in quantitative analysis performed by LC-MS/MS under the conditions described above.

Pro-Hyp: 12,772 ppm, Hyp-Gly: 6,353 ppm, Gly-Pro-Hyp: 32,010 ppm, Ala-Hyp-Gly: 454 ppm, Glu-Hyp-Gly: 24 ppm, Ser-Hyp-Gly: 239 ppm, Gly-Pro: 26,387 ppm, Pro-Ala-Gly: 2,183 ppm, total: 80,422 ppm.

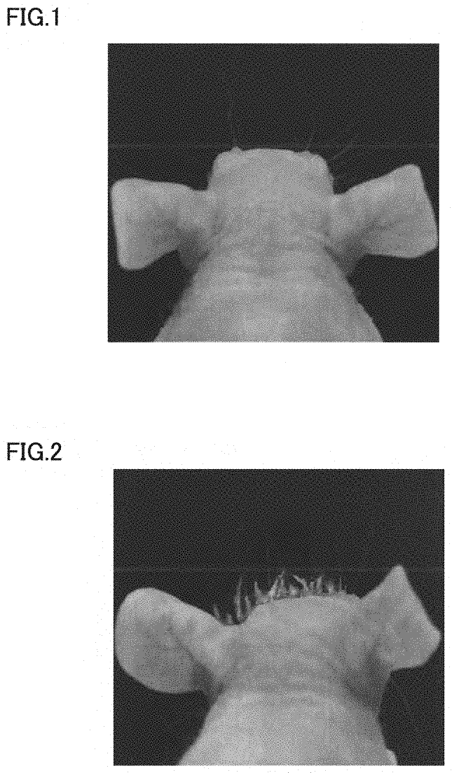

The heads of the hairless mice in the above-described groups were observed immediately after they were reared for 3 weeks to 11 weeks of age under the following conditions: the temperature was 23±2° C., the relative humidity was 55±10%, the lighting cycle was 12 hours, the light period started at 7:00 and ended at 19:00, and the mice were allowed to freely eat. Here, it is known that a hairless mouse starts to lose its hair after 2 weeks of age and has no hair at about 4 weeks of age. At the time of obtaining the hairless mice, their heads had no hair. FIG. 1 shows the head of a 11-week-old hairless mouse in the control group given the magnesium-deficient specialty feed. FIG. 2 shows the head of a 11-week-old hairless mouse in the first group given the magnesium-deficient specialty feed containing Pro-Hyp.

Resultantly, as is understood from comparison between FIGS. 1 and 2, hair development occurred in the hair of head in the hairless mice eating the mixed feed containing Pro-Hyp.

Further, a 2-week-old hairless mouse before starting to lose its hair was obtained from Hoshino Laboratory Animals, Inc., and relative to the amount of head hair of the hairless mouse which is defined as 10, the amount of head hair of each of the 11-week-old hairless mice in each group was visually measured. Table 3 shows the results. Each of the values in the table represents an average of the amounts of head hair of the six hairless mice in each group.

### [Table 3]

TABLE 3

| Group | Score |
|---|---|
| Normal group | 0 |
| Control group | 0 |
| First group | 8 |
| Second group | 3 |
| Third group | 7 |

### [Discussions]

The above-described results indicate that Pro-Hyp, the collagen peptide mixture A and the collagen peptide mixture C had a hair papilla cell growth promoting action, and were therefore effective as hair growing agents, specifically cell growth promoters for hair papilla cells, promoters of hair development or hair growth in the hair of head, or hair loss progression preventing agents.

### Example 3

### Control Test (Cell Biological Test: In Vitro Test)

### [Preparation of Sample]

### <Preparation of Amino Acid, Peptide and Collagen Peptide Mixture>

As samples to be used for evaluating the hair papilla cell growth promoting action, alanine (trade name: “L-Alanine”, manufactured by Kanto Kagaku Co., Inc., Catalog No: 01101-30), arginine (trade name: “L-Arginine”, manufactured by FUJIFILM Wako Pure Chemical Corporation, Catalog No: 015-04613), glutamine (trade name: “L-Glutamine”, manufactured by FUJIFILM Wako Pure Chemical Corporation, Catalog No: 074-00522) and proline (trade name: “L-Proline”, manufactured by FUJIFILM Wako Pure Chemical Corporation, Catalog No: 161-04602) were prepared.

### <Preparation of Hair Papilla Cells>

Hair papilla cells were prepared by the same method as described in the section <Preparation of Hair Papilla Cells>in Example 1 above.

### [Cell Growth Test]

The hair papilla cell growth promoting action in each sample (amino acid) and the significance were evaluated in the same manner as described in the section [Cell Growth Test] in Example 1 above. Table 4 shows the results. In any of the samples, a significant hair papilla cell growth promoting action was not exhibited.

TABLE 4

| | | | | Ratio to | |
|---|---|---|---|---|---|
| Amino acid | Content (final | | Growth rate | control t- | |
| or peptide | concentration) | | control = 100 | test | Assessment |
| Ala | 0.05 | mM | 94 ± 1 | 0.00 | − |
| | 0.5 | mM | 100 ± 8 | 0.96 | − |
| | 5 | mM | 95 ± 1 | 0.01 | − |
| Arg | 0.05 | mM | 93 ± 6 | 0.14 | − |
| | 0.5 | mM | 98 ± 3 | 0.35 | − |
| | 5 | mM | 101 ± 2 | 0.40 | − |
| Gln | 0.05 | mM | 92 ± 2 | 0.01 | − |
| | 0.5 | mM | 94 ± 5 | 0.12 | − |
| | 5 | mM | 100 ± 4 | 0.91 | − |
| Pro | 0.05 | mM | 91 ± 4 | 0.03 | − |
| | 0.5 | mM | 93 ± 5 | 0.09 | − |
| | 5 | mM | 96 ± 1 | 0.02 | − |

### [Discussions]

From Table 4, it is indicated that alanine, arginine, glutamine and proline had a poor hair papilla cell growth promoting action, and only specific amino acids such as Hyp had a hair papilla cell growth promoting action.

While embodiments and Examples of the present invention have been described above, the configurations of the embodiments and Examples described above may be appropriately combined as originally envisioned.

The embodiments and Examples disclosed herein should be regarded as illustrative rather than limiting in any way. The scope of the present invention is given by the appended claims rather than the foregoing description, and all changes which fall within the range of the appended claims and equivalents thereof are intended to be embraced therein.

1. A hair growing agent comprising one or more amino acids or peptides selected from the group consisting of Hyp, Pro-Hyp, Hyp-Gly, Gly-Pro, Leu-Hyp, Phe-Hyp, Pro-Ala, Pro-Gly, Pro-Pro, Glu-Hyp, Gly-Pro-Hyp, Ala-Hyp-Gly, Glu-Hyp-Gly, Pro-Ala-Gly and Ser-Hyp-Gly, a salt thereof, or a chemically modified product thereof.

2. The hair growing agent according to claim 1, wherein the amino acids and the peptides are derived from collagen.

3. The hair growing agent according to claim 1, wherein the hair growing agent is a collagen peptide mixture containing at least one of the amino acids or the peptides.

4. The hair growing agent according to claim 3, wherein the collagen peptide mixture has a weight average molecular weight of 100 Da or more and 8,000 Da or less.

5. The hair growing agent according to claim 1, wherein the hair growing agent is a cell growth promoter for hair papilla cells.

6. The hair growing agent according to claim 1, wherein the hair growing agent is a promoter of hair development or hair growth in the hair of head, or a hair loss progression preventing agent.

7. A food or beverage product comprising the hair growing agent according to claim 1.
